(19) United States
(12) Patent Application Publication
(10) Pub. No.: US 2009/0020732 A1
(43) Pub. Date: Jan. 22, 2009

(54) METHOD OF SELECTIVELY SEPARATING CARBON NANOTUBES, ELECTRODE COMPRISING METALLIC CARBON NANOTUBES SEPARATED BY THE METHOD AND OLIGOMER DISPERSANT FOR SELECTIVELY SEPARATING CARBON NANOTUBES

(75) Inventors: Hyeon-Jin SHIN, Yongin-si (KR); Jae-young CHOI, Yongin-si (KR); Seong-jae CHOI, Yongin-si (KR); Seon-mi YOON, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/123,015

(22) Filed: May 19, 2008

(30) Foreign Application Priority Data
May 29, 2007 (KR) 10 2007 0052220

(51) Int. Cl. D01F 9/12; C07D 409/02; C07D 409/14; H01B 1/22; C07D 333/06

(52) U.S. Cl. 252/503; 423/447.1; 549/59; 549/83; 977/845; 977/742; 977/750; 977/752

(57) ABSTRACT

Provided is method of selectively separating carbon nanotubes into metallic carbon nanotubes and semiconducting carbon nanotubes, the method including: preparing a mixture including a dispersant, carbon nanotubes, and a solvent; dispersing the carbon nanotubes in the mixture; and separating the semiconducting carbon nanotubes from the mixture in which the carbon nanotubes are dispersed, wherein the dispersant is an oligomer including about 2 to about 24 repeat units, each including a head moiety and a tail moiety, wherein the head moiety comprises 1 to about 5 aromatic hetero rings, and the tail moiety comprises a hydrocarbon chain or chains connected to the head moiety.

This application claims priority to Korean Patent Application No. 10-2007-0052220, filed on May 29, 2007, in the Korean Intellectual Property Office, and all the benefits accruing therefrom under 35 U.S.C. §119, the contents of which is incorporated herein in its entirety by reference.

### BACKGROUND OF THE INVENTION

This disclosure relates to a method of selectively separating carbon nanotubes into metallic carbon nanotubes and semiconducting carbon nanotubes, a display electrode including metallic carbon nanotubes separated by the method, and an oligomer dispersant for selectively separating carbon nanotubes.

Carbon nanotubes (CNT) are tubular materials made up of carbon atoms organized in a hexagonal honeycomb structure that have highly anisotropic properties. Carbon nanotubes can be in the form of single-walled carbon nanotubes and multi-walled carbon nanotubes. Single wall carbon nanotubes display roping, brought on by vander Waals forces that come into force between two neighboring single wall carbon nanotubes. Carbon nanotubes are a nanoscale material having diameters that are on the order of nanometers (nm) (1 nm=10⁻⁹ m). Carbon nanotubes have desirable properties such as excellent mechanical characteristics, electrical selectivity, excellent field emission characteristics, high efficient hydrogen storage characteristics, and the like.

In addition, carbon nanotubes may have metallic or semiconducting characteristics depending upon the arrangements of the carbon atoms and have various energy gaps according to the diameter of the tube. They also display a peculiar quantum effect because of a semi one-dimensional energy structure.

Methods of synthesizing carbon nanotubes include arc discharge, thermal decomposition, laser deposition, plasma chemical vapor deposition, thermal chemical vapor deposition, and electrolysis.

Carbon nanotubes having high electrical conductivity have been used to form a conductive film, and the possibility of using carbon nanotubes in various applications such as a field emission display (FED) or a probe of a scanning probe microscope (SPM) is increasing. Accordingly, research on the use of carbon nanotubes has been actively performed.

Carbon nanotubes need to be separated into metallic carbon nanotubes and semiconducting carbon nanotubes in order to use them effectively. For example, metallic carbon nanotubes may be used to form an electrode to obtain high conductivity, and semiconducting carbon nanotubes may be used in a semiconductor layer of a transistor.

As a result, there has been considerable research on separating carbon nanotubes.

Synthetic Metals, 2001, 121, 1211 discloses a method of separating carbon nanotubes through electrophoresis after the carbon nanotubes are dispersed using a dispersant. Science, 2003, 301, 1519 discloses a method of separating carbon nanotubes by adding a functional group to metallic carbon nanotubes having low band gaps, rather than semiconducting carbon nanotubes having large band gaps.

However, on disadvantage of both methods is that they cannot separate carbon nanotubes having various diameters but only metallic carbon nanotubes having small diameters.

In addition, Nano Letters 2003, 3, 1245 discloses a method of separating metallic carbon nanotubes by promoting adsorbtion of Br₂ by the metallic carbon nanotubes and then precipitating the metallic carbon nanotubes by their increased weight. Nature Materials 2003, 2, 338 discloses a method of separating carbon nanotubes by wrapping them with DNA and then using an ion exchange column to separate them. Nature Nanotechnology 2006, 1, 60 discloses a method of separating carbon nanotubes by dispersing the carbon nanotubes using a biopolymer and separating the carbon nanotubes according to their weight based on the fact that the weight of a carbon nanotube varies according to its diameter. However, these methods can only be realized when carbon nanotubes have large weight differences. Current Applied Physics 2006, 6S1, e99 discloses a method of separating out metallic carbon nanotubes by dissolving them in a solution.

However, these methods are disadvantageous in that these methods only differentiate on the basis of size, and they cannot separate metallic carbon nanotubes from semiconducting carbon nanotubes.

Carbon nanotubes prepared according to conventional preparation methods have both metallic and semiconducting characteristics, and these two types of carbon nanotubes cannot be easily separated. It is therefore desirable to have a method of efficiently separating carbon nanotubes into metallic carbon nanotubes and semiconducting carbon nanotubes.

### SUMMARY OF THE INVENTION

Disclosed herein is a method of selectively separating metallic carbon nanotubes from semiconducting carbon nanotubes. The method is efficient.

Disclosed herein too is a display electrode that comprises metallic carbon nanotubes that are separated by the method.

Disclosed herein too is an oligomer dispersant for selectively separating carbon nanotubes.

Disclosed herein too is a method of selectively separating carbon nanotubes into metallic carbon nanotubes and semiconducting carbon nanotubes, the method comprising preparing a mixture comprising a dispersant; carbon nanotubes; and a solvent; dispersing the carbon nanotubes in the mixture; and separating the semiconducting carbon nanotubes from the mixture in which the carbon nanotubes are dispersed, wherein the dispersant is an oligomer comprising 2 to 24 repeat units, each oligomer comprising a head moiety and a tail moiety, wherein the head moiety comprises 1 to 5 aromatic hetero rings, and the tail moiety comprises one or more hydrocarbon chains covalently bonded to the head moiety.

Disclosed herein too is a display electrode comprising metallic carbon nanotubes separated by the method.

Disclosed herein too is an oligomer dispersant for carbon nanotubes comprising 2 to 24 repeat units, each comprising a head moiety and a tail moiety, wherein the head moiety comprises 1 to 5 aromatic hetero rings, and the tail moiety comprises one or more hydrocarbon chains connected to the head moiety.

### BRIEF DESCRIPTION OF THE DRAWINGS

The above and other features and advantages of the present invention will become more apparent by describing in detail exemplary embodiments thereof with reference to the attached drawings in which:

FIG. 1 shows a graph illustrating UV-Vis-NIR Spectroscopy properties of carbon nanotube solutions according to the types of dispersants; and

FIG. 2 shows a normalized graph of FIG. 1 by revising base lines.

### DETAILED DESCRIPTION OF THE INVENTION

The present invention will now be described more fully with reference to the accompanying drawings in which exemplary embodiments are shown.

A conventional method of separating carbon nanotubes uses a dispersant that does not have any particular selectivity for metallic carbon nanotubes over semiconducting carbon nanotubes. Disclosed herein is a method of selectively separating carbon nanotubes that uses a dispersant having a high selectivity for metallic carbon nanotubes over semiconducting carbon nanotubes. Once the dispersant is mixed with the carbon nanotubes, the metallic carbon nanotubes and the semiconducting carbons can be separated with a high yield.

A method of selectively separating carbon nanotubes into metallic carbon nanotubes and semiconducting carbon nanotubes includes preparing a mixture including a dispersant; carbon nanotubes; and a solvent; dispersing the carbon nanotubes in the mixture; and separating the semiconducting carbon nanotubes from the mixture in which the carbon nanotubes are dispersed. The dispersant is an oligomer comprising about 2 to about 24 repeat units, each comprising a head moiety and a tail moiety, wherein the head moiety includes 1 to about 5 aromatic hetero rings, and the tail moiety includes one or more hydrocarbon chains connected to the head moiety.

Herein, the terminology “oligomer” indicates a compound including a plurality of repeat units in one molecule with a number average molecular weight of less than 5000 grams per mole, and the terminology “polymer” indicates a compound including a plurality of repeat units in one molecule with a number average molecular weight of greater than 5000 grams per mole.

In preparing a mixture, the order of adding the dispersant and the carbon nanotubes into the solvent is not critical. Generally, a sonicator is used to disperse the carbon nanotubes in the mixture. However, any method that can destroy agglomeration of the carbon nanotubes can be used. The carbon nanotubes may be dispersed using the sonicator for about 1 to about 20 hours, and preferably for about 10 hours. Finally, separating the semiconducting carbon nanotubes from the mixture in which the carbon nanotubes are dispersed may be performed, for example, by running the mixture through a centrifuge and separating a filtrate which mainly contains the semiconducting carbon nanotubes from a precipitate which mainly contains the metallic carbon nanotubes.

An oligomer dispersant which includes about 2 to about 24 repeat units, each oligomer including a head moiety having 1 to about 5 aromatic hetero rings and a tail moiety having one or more hydrocarbon chains covalently bonded to the head moiety displays a very high selectivity for separating the metallic carbon nanotubes from the semiconducting carbon nanotubes.

The number of the aromatic hetero rings in the dispersant used in the method of selectively separating carbon nanotubes may be about 7 to about 24. Less than 7 aromatic hetero rings may result in reduction in selectivity. On the other hand, more than 24 aromatic hetero rings may result in an increase of the molecular weight of the dispersant, and thus a large amount of dispersant may be required to disperse the carbon nanotubes.

In addition, the number of the aromatic hetero rings in the head moiety of a repeat unit in the oligomer dispersant may be about 2 to about 5. Since one repeat unit includes one hydrocarbon chain, the ratio of the aromatic hetero ring to the hydrocarbon chain is about 2:1 to about 5:1. The dispersant having at least two aromatic hetero rings has higher selectivity than one having a single aromatic hetero ring. However, more than 5 aromatic hetero rings may decrease dispersion stability since the size of the tail moiety is smaller than that of the head moiety.

Further, the hydrocarbon chain or chains of the tail moiety of the dispersant may be regioregularly arranged in one direction. For example, the regioregular arrangement indicates an arrangement in which a substituent is substituted at a particular site of the aromatic hetero rings of the head moiety in the repeat unit of the oligomer. In contrast, the regiorandom arrangement indicates an arrangement in which the hydrocarbon chain or chains of the tail moiety are randomly substituted at the aromatic hetero rings of the head moiety. Accordingly, the hydrocarbon chain or chains of the tail moiety may be regiorandomly arranged in various directions.

In a dispersant having the same chemical formula, selectivity of a dispersant having regioregular arrangement is greater than that of a dispersant having regiorandom arrangement since the tail moiety tilts because of steric force in regiorandom structure and adsorption of the head moiety to the carbon nanotubes may decrease.

The oligomer dispersant may be a compound represented by one of Formulae 1 through 10 below.

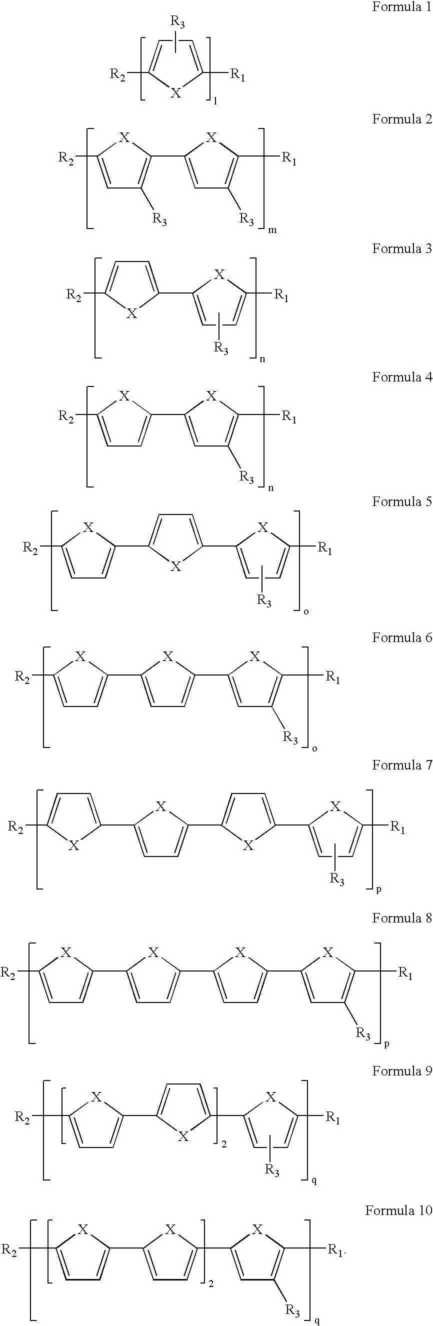

Here, R₁ and R₂ are each independently a hydrogen atom or a C1-C3 alkyl group, R₃ is a C5-C10 alkyl group, X is S, O or NH, I is an integer of about 8 to about 24, m and n are each independently an integer of about 4 to about 12, o is an integer of about 3 to about 8, p is an integer of about 2 to about 6, and q is an integer of about 2 to about 4, and preferably X in Formulae 1 through 10 is S.

Here, substitution sites of R₃ vary in at least one adjacent repeat unit in Formulae 1, 3, 5, 7 and 9.

Preferably, the dispersant may be a compound represented by one of Formulae 11 through 22 below.

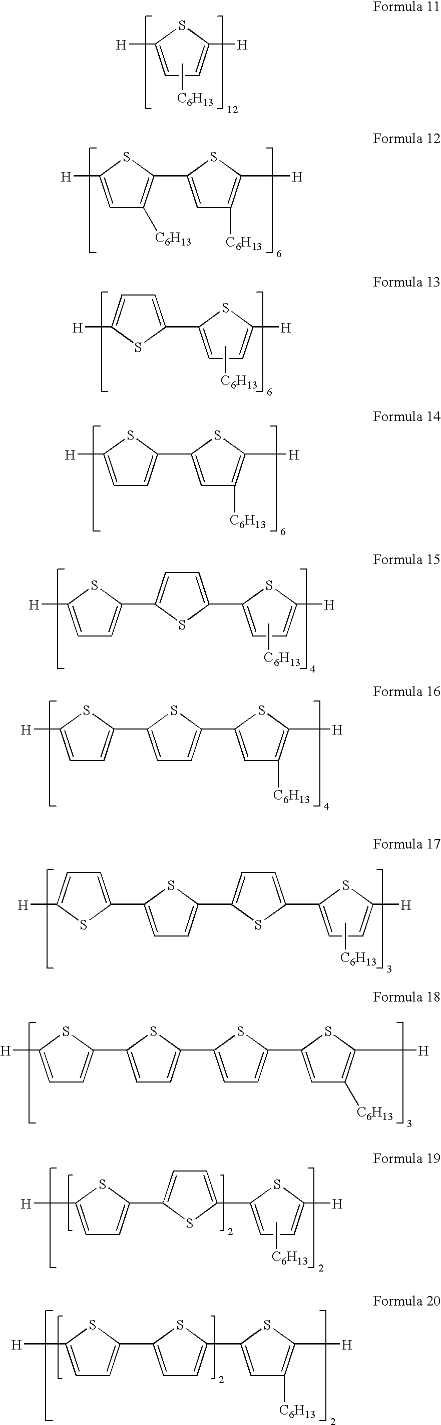

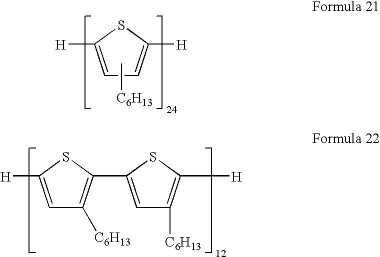

Here, substitution sites of C₆H₁₃ vary in at least one adjacent repeat unit in Formulae 11, 13, 15, 17, 19 and 21.

The solvent may be an organic solvent, water, or a mixture thereof, but is not limited thereto.

The mixture may include about 0.01 to about 1 wt % of the dispersant, about 0.01 to about 0.1 wt % of the carbon nanotubes, and remaining wt % of the solvent based on 100 wt % of the mixture.

In addition, the weight ratio between the carbon nanotubes and the dispersant may be in the range of about 1:1 to about 1:10. When the amount of the dispersant is less than the range described above, the carbon nanotubes cannot be sufficiently dispersed. On the other hand, when the amount of the dispersant is greater than the range described above, effects may decrease because of the viscosity of the dispersant itself.

The carbon nanotubes may be at least one selected from the group consisting of single-walled carbon nanotubes, double-walled carbon nanotubes, multi-walled carbon nanotubes and rope-like carbon nanotubes. However, any type of carbon nanotubes that are used in the art can be used.

The organic solvent is at least one selected from the group consisting of alcohols such as methyl alcohol, ethyl alcohol, n-propyl alcohol, isopropyl alcohol, n-butyl alcohol, sec-butyl alcohol, t-butyl alcohol, isobutyl alcohol, and diacetone alcohol; ketones such as acetone, methylethyl ketone, and methylisobutyl ketone; glycols such as ethylene glycol, diethylene glycol, triethylene glycol, propylene glycol, butylene glycol, hexylene glycol, 1,3-propandiol, 1,4-butanediol, 1,2,4-butanetriol, 1,5-pentanediol, and 1,2-hexanediol, 1,6-hexanediol; glycol ethers such as ethylene glycol monomethyl ether, triethylene glycol monoethyl ether; glycol ether acetates such as propylene glycol monomethyl ether acetate (PGMEA); acetates such as ethyl acetate, butoxyethoxy ethyl acetate, butyl carbitol acetate (BCA), and dihydroterpineol acetate (DHTA); terpineols; a halogen containing solvent, a nitrogen containing solvent; trimethyl pentanediol monoisobutyrate (TEXANOL); dichloroethene (DCE); chloroform, dichlorobenzene, iodobenzene, nitromethane, nitroethane, acetonitrile, benzonitrile and 1-methylpyrrolidone (NMP).

The mixture used in the method of selectively separating carbon nanotubes can be applied in various industrial fields in which water or oil-based carbon nanotube compositions are used such as in manufacturing of emitters for field emission displays (FEDs), carbon nanotube ink, printable carbon nanotubes, and the like.

The metallic carbon nanotubes separated as described above can be advantageously used in a display electrode.

When separating the filtrate that mainly contains the dispersed semiconducting carbon nanotubes, a portion precipitated by a centrifuge includes mainly the metallic carbon nanotubes. In other words, the separated metallic carbon nanotubes are obtained in the precipitated portion. The metallic carbon nanotubes can be coated as a thin film on a display device in which it is desirable to have transparency and conductivity. It can also be used as a display electrode.

The present invention also provides an oligomer dispersant for carbon nanotubes comprising about 2 to about 24 repeat units, each having a head moiety and a tail moiety, wherein the head moiety includes 1 to about 5 aromatic hetero rings, and the tail moiety includes one or more hydrocarbon chains connected to the head moiety.

The number of the aromatic hetero ring in the dispersant may be in the range of about 7 to about 24, and the number of the aromatic hetero ring in the head moiety of each of the repeat units may be in the range of about 2 to about 5.

The hydrocarbon chain or chains of the tail moiety may be regioregularly arranged in one direction.

The dispersant may be a compound represented by one of Formula 1 to 10 below.

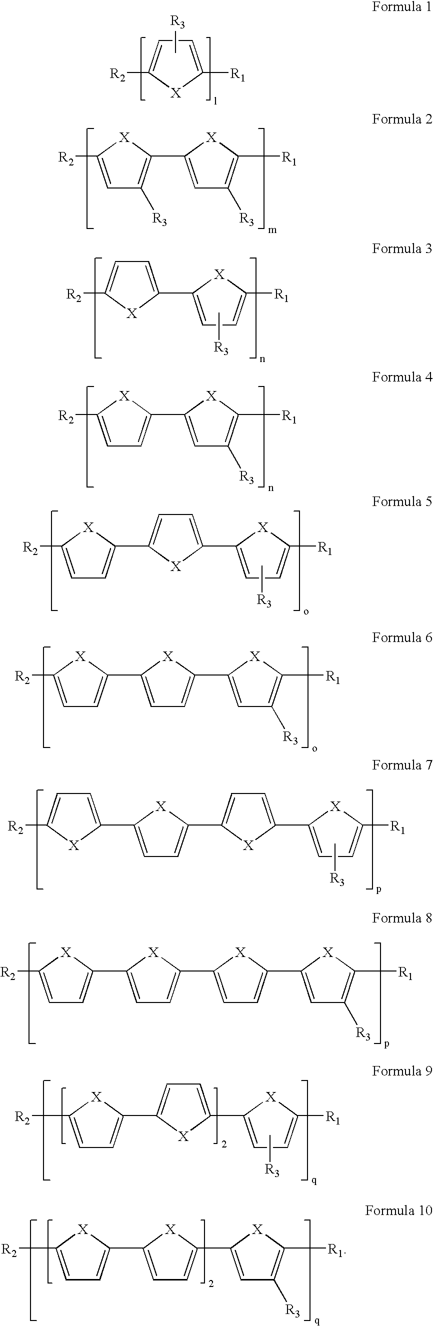

Here, R₁ and R₂ are each independently a hydrogen atom or a C1-C3 alkyl group, R₃ is a C5-C10 alkyl group, X is S, O or NH, I is an integer of about 8 to about 24, m and n are each independently an integer of about 4 to about 12, o is an integer of about 3 to about 8, p is an integer of about 2 to about 6, and q is an integer of about 2 to about 4. X in Formulae 1 through 10 is S.

Here, substitution sites of R₃ vary in at least one adjacent repeat unit in Formulae 1, 3, 5, 7 and 9.

Preferably, the dispersant may be a compound represented by one of Formulae 11 through 22 below.

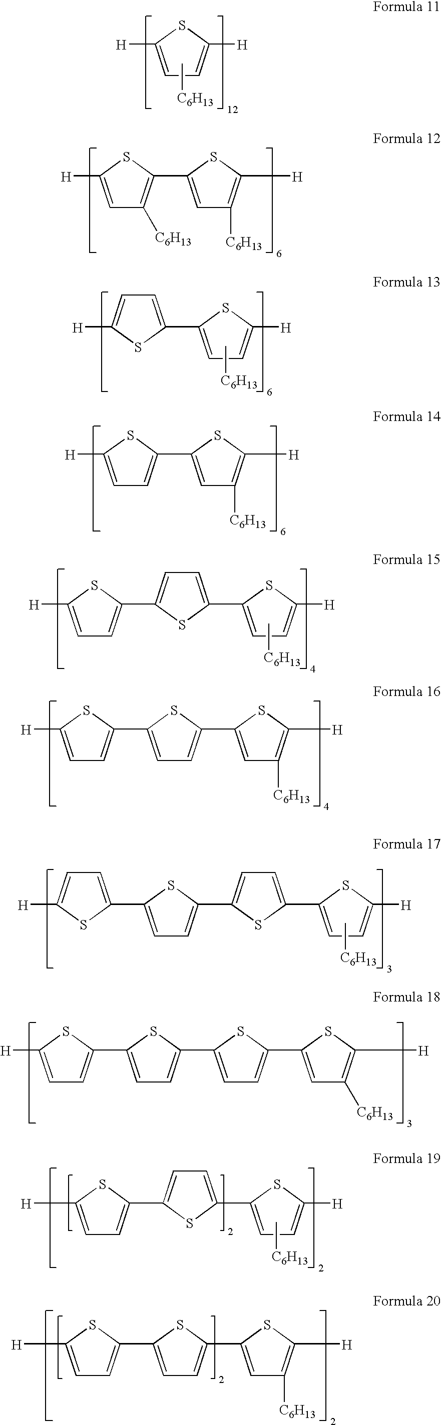

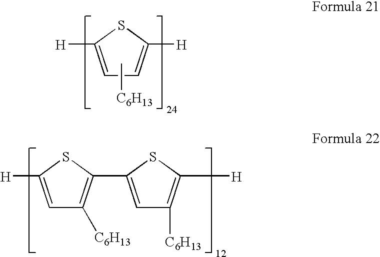

Here, substitution sites of C₆H₁₃ vary in at least one adjacent repeat unit in Formulae 11, 13, 15, 17, 19 and 21.

The present invention will now be described in greater detail with reference to the following examples. The following examples are for illustrative purposes only and are not intended to limit the scope of the invention.

### EXAMPLES

### Preparation Example 1

### Synthesis of Dispersant 1 and Dispersant 2

1.48 g (61.32 mmol) of Mg and 100 mL of tetrahydrofuran (THF) were added to a reactor. 25.3 mg (0.1 mmol) of I₂ was added to the reactor while heating the reactor, and then 20 g (61.32 mmol) of 2,5-dibromo-3-hexylthiophene was gradually added thereto and the mixture was refluxed until Mg was completely dissolved. Then, 0.96 g (1.83 mmol) of NiCl₂(dppp) (dppp=I,3-bis(diphenylphosphino)propane) was added to the reactor and the mixture was refluxed for about 5 hours. When the reactions were terminated, the mixture was cooled to room temperature to remove the solvent and the material remaining was subjected to extraction by ethylenedichloride (EDC). The solids not dissolved in EDC was filtered, and the solvent was removed from the remaining solution using an evaporator. Finally, the residue was dried in a vacuum to obtain a red solid. The obtained solid was subjected to extraction using a solvent extraction to obtain Dispersant 1 represented by Formula 23 and Dispersant 2 represented by Formula 12. Dispersants 1 and 2 are separated according to their molecular weights.

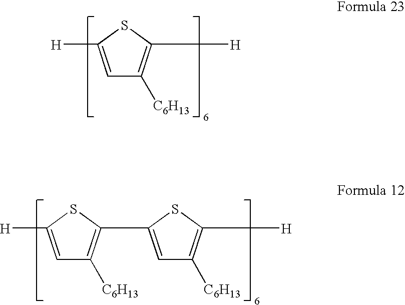

### Preparation Example 2

### Synthesis of Dispersant 3 and Dispersant 6

19.27 g (118.83 mmol) of FeCl₃ and 100 mL of chloroform (CHCl₃) were added to a reactor in an Ar atmosphere and the reactor was cooled to 0° C. 5 g (29.70 mmol) of 3-hexylthiophene was gradually added thereto and stirred at the same temperature for 30 minutes. After the reactions were terminated, the reactants were added to 800 mL of methanol and stirred for about 1 hour. Then, a solid generated in the reaction solution was filtered, and the filtered solid was added to 200 mL of 30% ammonium chloride solution. The mixture was stirred for about 1 hour and was subjected to extraction using EDC. A solid not dissolved in EDC was filtered, and the solvent was removed from the remaining solution using an evaporator. Finally, the remaining residue was dried in a vacuum to obtain a red solid. The obtained solid was subjected to extraction using a solvent extraction to obtain Dispersant 3 represented by Formula 11 and Dispersant 6 represented by Formula 21 according to their molecular weights.

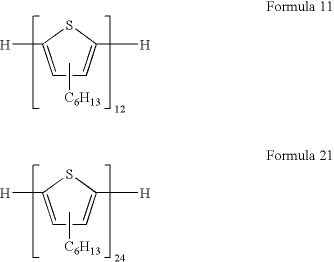

### Preparation Example 3

### Synthesis of Dispersant 4

### Synthesis of Intermediate 1

28 mL (300 mmol) of 3-bromothiophene and 0.8 g (1.5 mmol) of NiCl₂(dppp) were dissolved in 250 mL of ether, and hexylmagnesium bromide [8.75 g (360 mmol) of magnesium and 49.5 g (420 mmol) of hexyl bromide reacted in 250 mL of ether] was gradually added thereto at 0° C. Then the temperature was gradually raised to room temperature and the mixture was refluxed. After the reactions were terminated, an aqueous ammonium chloride solution was added to the reactor and the mixture was subjected to extraction using ether. The resultant was purified by vacuum distillation to obtain 41.0 g (244 mmol) of Intermediate 1 (Yield: 81%).

¹H NMR (δ, CDCl₃): 7.10 (d, 1H), 6.88 (d, 1H), 6.84 (s, 1H), 2.58 (t, 2H), 1.59 (m, 2H), 1.26 (m, 8H), 0.88 (m, 3.7H).

### Synthesis of Intermediate 2

41 g (244 mmol) of Intermediate 1 was added to 500 mL of THF, and cooled to −20° C., and then 40.21 mL (268 mmol) of N,N,N,N-tetramethyl-ethylenediamine was added thereto. After 30 minutes, the mixture was cooled to −78° C., 160 mL of n-butyllithium (1.6 M in hexane) was added thereto and the mixture was heated to room temperature and refluxed for 3 hours. The mixture was cooled again to −78° C., and 60 mL (293 mmol) of 2-isopropoxy-4,4,5,5-tetramethyl-1,3,2-dioxabororane was added thereto. Then, the temperature was gradually raised to room temperature and placed overnight at room temperature. Then, the mixture was subjected to extraction using H₂O and CH₂Cl₂, and isolated by silica gel column chromatography to obtain 50.3 g (171 mmol) of Intermediate 2 (Yield: 70%).

¹H NMR (δ, CDCl₃): 7.46 (s, 1H), 7.20 (s, 1H), 2.61 (t, 2H), 1.59 (m, 2H), 1.30 (m, 22H), 0.88 (t, 4H).

### Synthesis of Intermediate 3

Intermediate 3 was obtained in the same manner as in the synthesis of Intermediate 1 except that 2-thiohexylmagnesium was used instead of hexylmagnesium bromide.

### Synthesis of Intermediate 4

8 g (48 mmol) of Intermediate 3 was dissolved in 50 mL of chloroform and 150 mL of acetic acid (AcOH), and the mixture was cooled to 0° C. Then, 8.55 g (48 mmol) of N-bromosuccinimide (NBS) was gradually added in small quantities in the absence of light. The mixture was quenched using an aqueous Na₂SO₃ solution and was subjected to extraction using hexane. 7.4 g (30 mmol) of Intermediate 4 as a white solid was obtained by recrystallization (Yield: 63%).

### Synthesis of Intermediate 5

5.3 g (32 mmol) of 2-bromothiophene, 10.5 g (35.5 mmol) of Intermediate 2, 10.09 g (102 mmol) of K₂CO₃, 3.9 g (3.4 mmol) of Pd(PPh₃)₄ (PPh₃: triphenylphosphine) were added to 150 mL of dimethoxyethane and 100 mL of H₂O and refluxed. Then, an aqueous ammonium chloride solution was added thereto and the mixture was subjected to extraction using chloroform, and purified by vacuum distillation to obtain 6.1 g (24.4 mmol) of Intermediate 5 (Yield: 76%).

¹H NMR (δ, CDCl₃): 7.10 (m, 2H), 6.98 (d, 1H), 6.93 (dd, 1H), 6.73 (d, 1H), 2.56 (t, 2H), 1.59 (m, 2H), 1.26 (m, 8H), 0.88 (m, 4H).

### Synthesis of Dispersant 4

11.7 g (72 mmol) of anhydrous FeCl₃ was added to 150 mL of chloroform and cooled to 0° C. Then, 6 g (24 mmol) of Intermediate 5 was dissolved in 20 mL of chloroform was gradually added to the FeCl₃ solution. After the reactions were terminated, the mixture was added to methyl alcohol to be precipitated. Then, a solid obtained after the reaction solution was filtered was added to an aqueous NH₄OH solution and stirred at room temperature. If the solid was red, the solid was washed several times with methyl alcohol, and dried to obtain 4 g of Dispersant 4 represented by Formula 26.

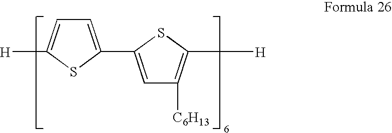

### Preparation Example 4

### Synthesis of Dispersant 5

### Synthesis of Intermediate 6

Intermediate 6 was synthesized in the same manner as in the synthesis of Intermediate 5, except that Intermediate 4 was used instead of 2-bromothiophene.

### Synthesis of Dispersant 5

Dispersant 5 represented by Formula 27 was synthesized in the same manner as in the synthesis of Dispersant 4 except that Intermediate 6 was used instead of Intermediate 5.

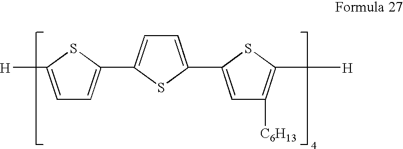

The Dispersants 4 and 5 are synthesized through schematic reaction schemes below.

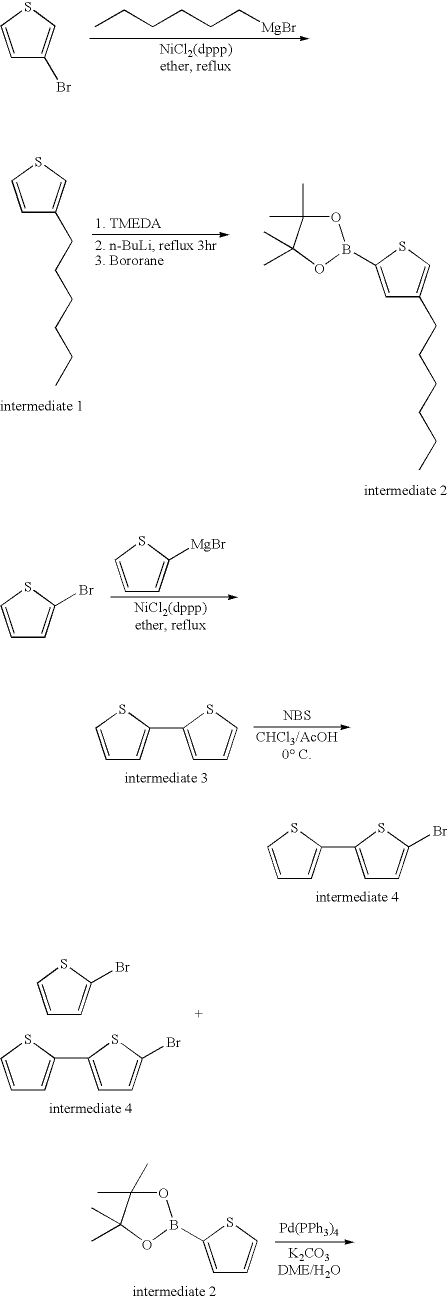

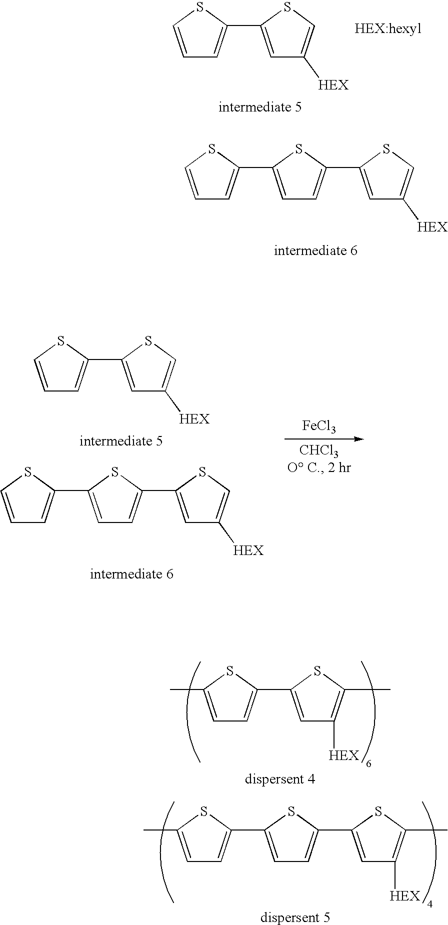

### Preparation of Isolated Carbon Nanotube Dispersion

### Comparative Example 1

6 mg of single-walled carbon nanotubes was added to 30 ml of o-dichlorobenzene (ODCB) not having the dispersant and dispersed in a sonicator for 10 hours. The dispersion was then centrifuged at 8,000 rpm for 10 minutes, and an isolated carbon nanotube dispersion was obtained by removing a precipitate.

### Comparative Example 2

3 mg of Dispersant 1 was dissolved in 30 ml of o-dichlorobenzene (ODCB), and 6 mg of single-walled carbon nanotubes was added to the solution, and then the mixture was dispersed in a sonicator for 10 hours. Then, the dispersion was centrifuged at 8,000 rpm for 10 minutes, and an isolated carbon nanotube dispersion was obtained by removing a precipitate.

### Comparative Example 3

An isolated carbon nanotube dispersion was obtained in the same manner as in Comparative Example 2, except that 30 mg of Dispersant 7 represented by Formula 28 was used instead of Dispersant 1.

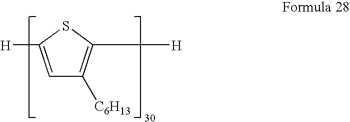

### Example 1

An isolated carbon nanotube dispersion was obtained in the same manner as in Comparative Example 2, except that 6 mg of Dispersant 2 was used instead of Dispersant 1.

### Example 2

An isolated carbon nanotube dispersion was obtained in the same manner as in Comparative Example 2, except that 6 mg of Dispersant 3 was used instead of Dispersant 1.

### Example 3

An isolated carbon nanotube dispersion was obtained in the same manner as in Comparative Example 2, except that 4.5 mg of Dispersant 4 was used instead of Dispersant 1.

### Example 4

An isolated carbon nanotube dispersion was obtained in the same manner as in Comparative Example 2, except that 4 mg of Dispersant 5 was used instead of Dispersant 1.

### Example 5

An isolated carbon nanotube dispersion was obtained in the same manner as in Comparative Example 2, except that Dispersant 8 represented by Formula 22 was used instead of Dispersant 1.

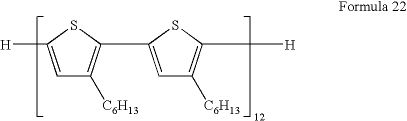

### Evaluation of Carbon Nanotube Separation Efficiency

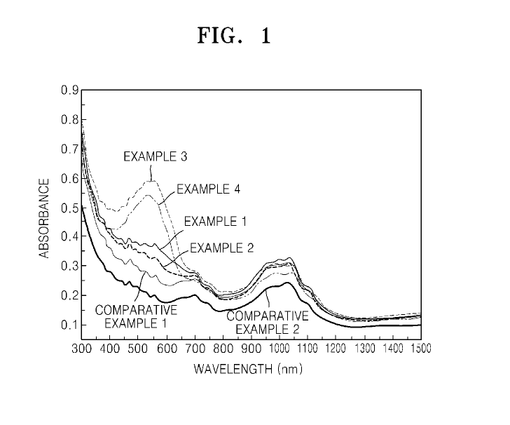

UV-Vis-NIR absorption spectra of the dispersions prepared according to Comparative Examples 1 to 3 and Examples 1 to 5 were measured, and the results of Comparative Examples 1 and 2 and Examples 1 to 4 are shown in FIG. 1.

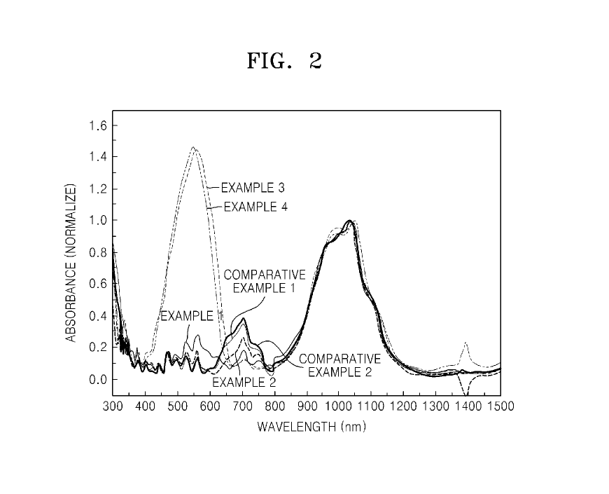

To facilitate comparison of the obtained spectra, base lines were revised and the graph of FIG. 1 was normalized in FIG. 2. A peak indicated as M11 in FIG. 2 shows characteristics of the metallic carbon nanotubes, and a peak indicated as S22 shows characteristics of the semiconducting carbon nanotubes.

Areas of each of the peaks were calculated, and the result is shown in Table 1 below.

The ratio of M11 is calculated using the equation below.

Amount of metallic carbon nanotubes(%)=Area of M11/(Area of M11+Area of S22)×100

TABLE 1

| | | | Amount of | Amount of |
|---|---|---|---|---|
| | | | metallic | reduced |
| | Area of | Area of | carbon | carbon |
| | S22 | M11 | nanotubes (%) | nanotubes (%) |
| Comparative | 199.9 | 36.1 | 15.28 | — |
| Example 1 | | | | |
| Comparative | 205.3 | 40.1 | 16.33 | Increased (6.86%) |
| Example 2 | | | | |
| Comparative | 235.8 | 30.79 | 11.55 | 24.41 |
| Example 3 | | | | |
| Example 1 | 282.7 | 1.3 | 0.45 | 97.07 |
| Example 2 | 195.7 | 10.9 | 5.29 | 65.36 |
| Example 3 | 257.2 | 2.3 | 0.88 | 94.22 |
| Example 4 | 152.8 | 3.4 | 2.19 | 85.68 |
| Example 5 | 202.5 | 9.7 | 4.55 | 70.23 |

As shown in Table 1, the amount of the metallic carbon nanotubes in the dispersions in the Examples 1-5 have been drastically decreased compared with the amount shown for the Comparative Examples 1-3. Thus, Examples 1-5 show improved separation efficiency compared to Comparative Examples 1-3.

Even when the dispersants have similar structures, efficiency of a dispersant having more than 7 repeat units in separating the metallic carbon nanotubes and the semiconducting carbon nanotubes increases significantly as shown in the results of Example 1 and Comparative Example 2. That is, the amount of the metallic carbon nanotubes decreased greater than 65%.

Further, even when the dispersants have similar structures, efficiency of a dispersant having more than 24 repeat units in separating the metallic carbon nanotubes and the semiconducting carbon nanotubes largely decreased as shown in the results of Example 5 and Comparative Example 3.

The dispersant having less than 6 repeat units had lower separation efficiency compared to the efficiency of not using the dispersant. In fact, the amount of the metallic carbon nanotubes increased.

As shown in the results of Examples 2, 3 and 4, when the number of aromatic hetero rings in the head moiety is the same, the separation efficiency largely increased when the ratio of the aromatic hetero rings in the head moiety to the hydrocarbon chain or chains in the tail moiety is greater than 2:1 compared to when the ratio of the aromatic hetero ring in the head moiety to the hydrocarbon chain or chains in the tail moiety is 1:1. That is, when the ratio of the aromatic hetero ring in the head moiety to the hydrocarbon chain or chains in the tail moiety is greater than 2:1, the amount of the metallic carbon nanotubes decreased by more than 85%.

Further, comparing Example 1 and Example 2 demonstrates that the aromatic hetero ring having stereoregular structure had highly increased separation efficiency compared with the aromatic hetero ring having stereorandom structure. That is, in the stereoregular structure, the amount of the metallic carbon nanotubes decreased by more than 97%.

These results show that the using the method can separate carbon nanotubes into metallic carbon nanotubes and semiconducting carbon nanotubes with a high yield. In particular, when the dispersant has stereoregularity, increased separation efficiency can be obtained, particularly, when the ratio of the aromatic hetero ring in the head moiety to the hydrocarbon chain or chains in the tail moiety is greater than 2:1 and when the number of repeat units in the dispersant is in the range of 7 to 24.

According to the method of selectively separating carbon nanotubes of the present invention, the metallic carbon nanotubes and the semiconducting carbon nanotubes can be efficiently separated using an oligomer dispersant having high selectivity to the metallic carbon nanotubes and the semiconducting carbon nanotubes.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of selectively separating carbon nanotubes into metallic carbon nanotubes and semiconducting carbon nanotubes, the method comprising:
preparing a mixture comprising a dispersant, carbon nanotubes, and a solvent;
dispersing the carbon nanotubes in the mixture; and
separating the semiconducting carbon nanotubes from the mixture in which the carbon nanotubes are dispersed; wherein
the dispersant is an oligomer comprising about 2 to about 24 repeat units, each oligomer comprising a head moiety and a tail moiety; wherein
the head moiety comprises 1 to about 5 aromatic hetero rings; and
the tail moiety comprises one or more hydrocarbon chains covalently bonded to the head moiety.

2. The method of claim 1, wherein the number of the aromatic hetero rings in the dispersant is in the range of about 7 to about 24.

3. The method of claim 1, wherein the number of the aromatic hetero rings in the head moiety of each of the repeat units is in the range of about 2 to about 5.

4. The method of claim 1, wherein the hydrocarbon chain or chains of the tail moiety are regioregularly arranged in one direction.

5. The method of claim 1, wherein the dispersant is a compound selected from the group consisting of Formulae 1 through 10 below: 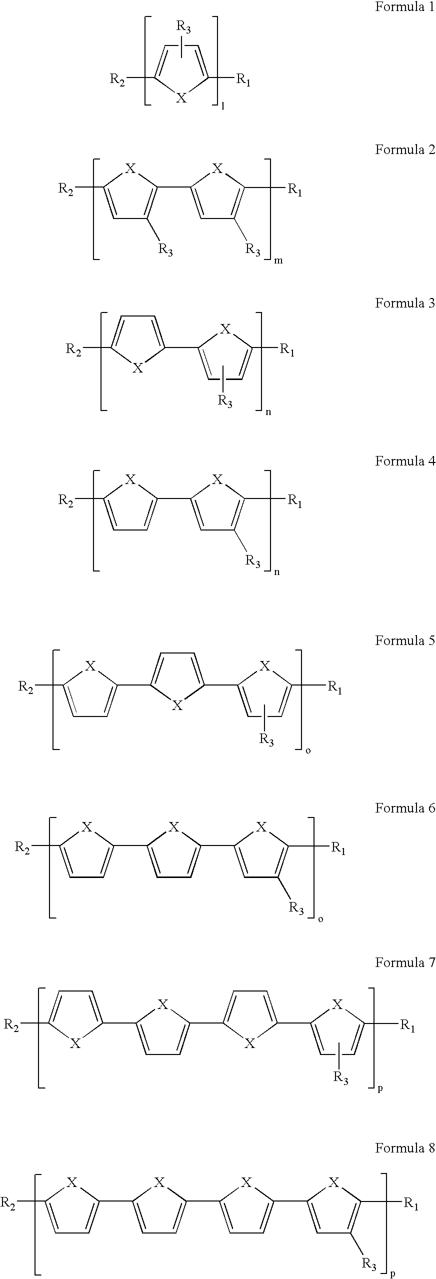 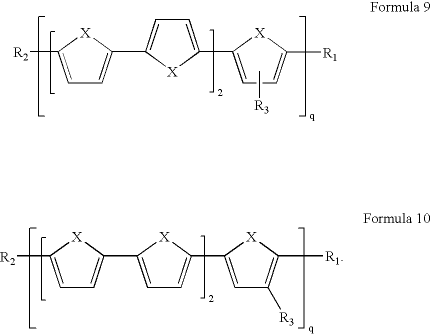
where R₁ and R₂ are each independently a hydrogen atom or a C1-C3 alkyl group, R₃ is a C5-C10 alkyl group, X is S, O or NH, I is an integer of about 8 to about 24, m and n are each independently an integer of about 4 to about 12, o is an integer of about 3 to about 8, p is an integer of about 2 to about 6, and q is an integer of about 2 to about 4, and wherein substitution sites of R₃ vary in at least one adjacent repeat unit in Formulae 1, 3, 5, 7 and 9.

6. The method of claim 5, wherein X in Formulae 1 through 10 is S.

7. The method of claim 1, wherein the dispersant is a compound represented by one selected from the group consisting Formulae 11 through 22 below: 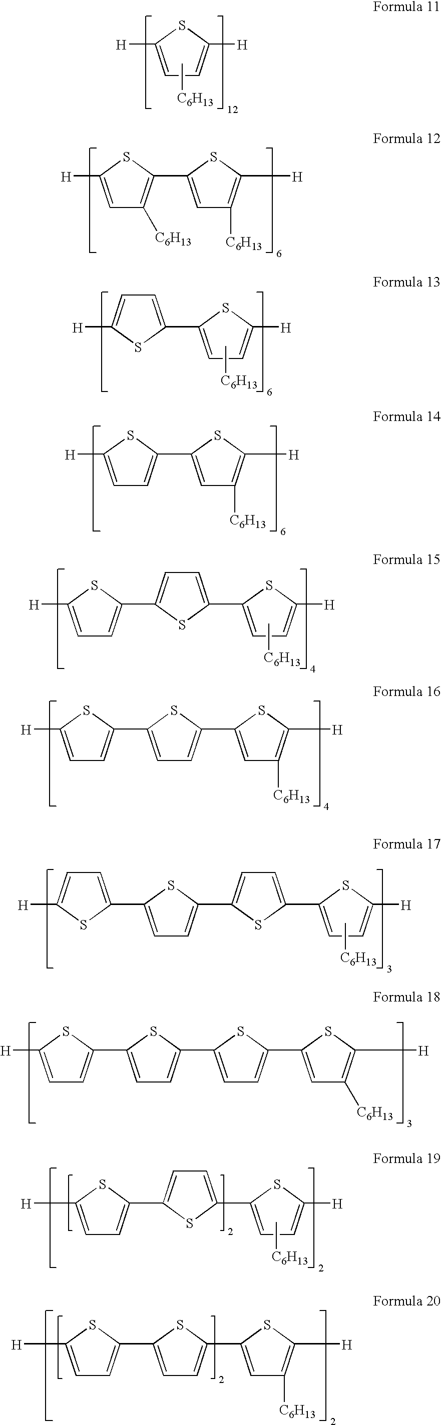 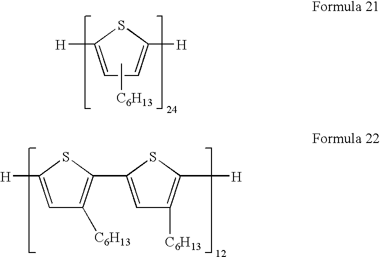
and wherein substitution sites of C₆H₁₃ vary in at least one adjacent repeat unit in Formulae 11, 13, 15, 17, 19 and 21.

8. The method of claim 1, wherein the solvent is an organic solvent, water, or a mixture thereof.

9. The method of claim 1, wherein the mixture comprises about 0.01 to about 1 wt % of the dispersant, about 0.01 to about 0.1 wt % of the carbon nanotubes, with the remainder being solvent, based on 100 wt % of the mixture.

10. The method of claim 1, wherein the weight ratio between the carbon nanotubes and the dispersant is in the range of about 1:1 to about 1:10.

11. The method of claim 1, wherein the carbon nanotubes are selected from the group consisting of single-walled carbon nanotubes, double-walled carbon nanotubes, multi-walled carbon nanotubes, roped carbon nanotubes and carbon nanotube bundles.

12. The method of claim 1, wherein the organic solvent is selected from the group consisting of alcohols, methyl alcohol, ethyl alcohol, n-propyl alcohol, isopropyl alcohol, n-butyl alcohol, sec-butyl alcohol, t-butyl alcohol, isobutyl alcohol, diacetone alcohol, ketones, acetone, methylethyl ketone, methylisobutyl ketone, glycols ethylene glycol, diethylene glycol, triethylene glycol, propylene glycol, butylene glycol, hexylene glycol, 1,3-propandiol, 1,4-butanediol, 1,2,4-butanetriol, 1,5-pentanediol, 1,2-hexanediol, 1,6-hexanediol, glycol ethers, ethylene glycol monomethyl ether, triethylene glycol monoethyl ether; glycol ether acetates, propylene glycol monomethyl ether acetate (PGMEA); acetates, ethyl acetate, butoxyethoxy ethyl acetate, butyl carbitol acetate, dihydroterpineol acetate; terpineols; a halogen containing solvent, a nitrogen containing solvent; trimethyl pentanediol monoisobutyrate; dichloroethene; chloroform, dichlorobenzene, iodobenzene, nitromethane, nitroethane, acetonitrile, benzonitrile and 1-methylpyrrolidone.

13. A display electrode comprising metallic carbon nanotubes separated by the method of claim 1.

14. An dispersant for carbon nanotubes comprising:
an oligomer having about 2 to about 24 repeat units, each oligomer comprising a head moiety and a tail moiety, wherein
the head moiety comprises 1 to 5 aromatic hetero rings, and
the tail moiety comprises one or more hydrocarbon chains covalently bonded to the head moiety.

15. The oligomer dispersant for carbon nanotubes of claim 14, wherein the number of the aromatic hetero rings in the dispersant is in the range of about 7 to about 24.

16. The oligomer dispersant for carbon nanotubes of claim 14, wherein the number of the aromatic hetero rings in the head moiety of each of the repeat units is in the range of about 2 to about 5.

17. The oligomer dispersant for carbon nanotubes of claim 14, wherein the hydrocarbon chain or chains of the tail moiety is regioregularly arranged in one direction.

18. The oligomer dispersant for carbon nanotubes of claim 14, wherein the dispersant is a compound represented by one selected from the group consisting of Formulae 1 through 10 below: 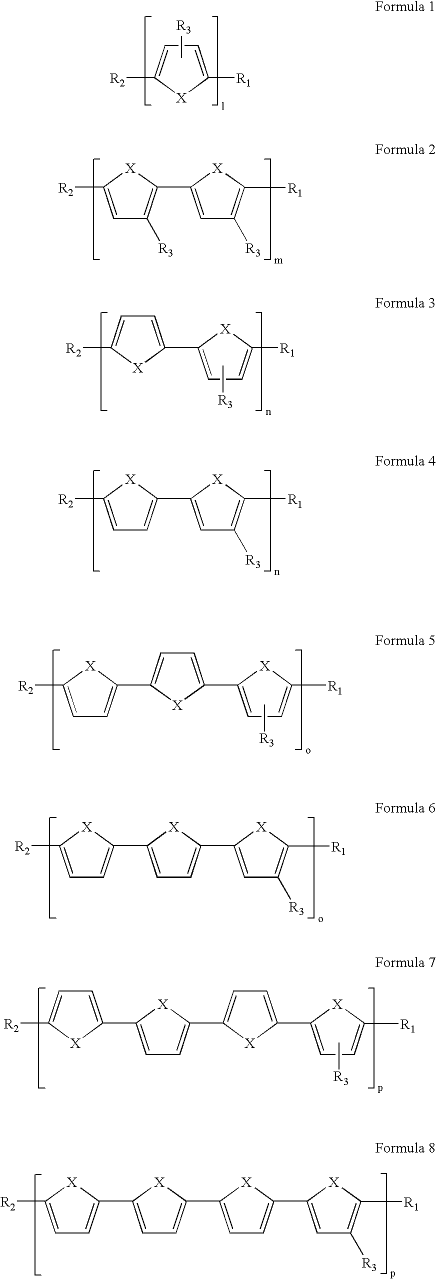 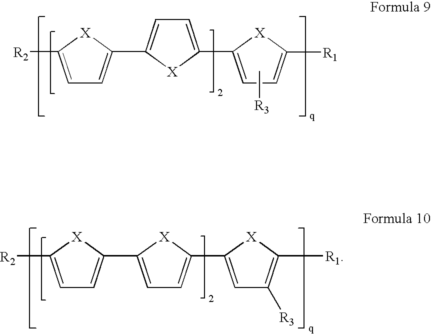
where R₁ and R₂ are each independently a hydrogen atom or a C1-C3 alkyl group,
R₃ is a C5-C10 alkyl group,
X is S, O or NH, I is an integer of about 8 to about 24, m and n are each independently an integer of about 4 to about 12, o is an integer of about 3 to about 8, p is an integer of about 2 to about 6, and q is an integer of about 2 to about 4, and wherein substitution sites of R₃ vary in at least one adjacent repeat unit in Formulae 1, 3, 5, 7 and 9.

19. The oligomer dispersant for carbon nanotubes of claim 18, wherein X in Formulae 1 through 10 is S.

20. The oligomer dispersant for carbon nanotubes of claim 14, wherein the dispersant is a compound represented by one selected from the group consisting of Formulae 11 through 22 below: 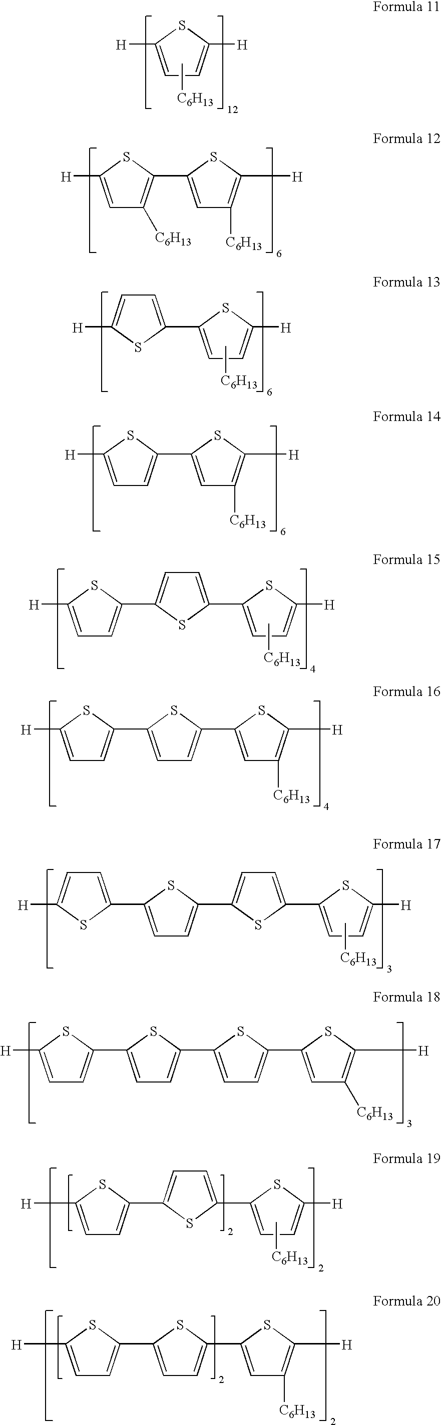 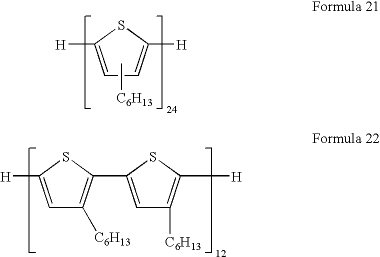
and wherein substitution sites of C₆H₁₃ vary in at least one adjacent repeat unit in Formulae 11, 13, 15, 17, 19 and 21.
